(19) United States
(12) Patent Application Publication
(10) Pub. No.: US 2023/0051627 A1
(43) Pub. Date: Feb. 16, 2023

(54) SINGLE-MOLECULE PROTEIN IDENTIFICATION VIA STRETCHING

(71) Applicant: PRESIDENT AND FELLOWS OF HARVARD COLLEGE, Cambridge, MA (US)

(72) Inventors: Mingjie DAI, Cambridge, MA (US); George M. CHURCH, Cambridge, MA (US); Marc W. KIRSCHNER, Cambridge, MA (US)

(73) Assignee: PRESIDENT AND FELLOWS OF HARVARD COLLEGE, Cambridge, MA (US)

(21) Appl. No.: 17/878,534

(22) Filed: Aug. 1, 2022

Related U.S. Application Data
(60) Provisional application No. 63/227,560, filed on Jul. 30, 2021.

(51) Int. Cl. G01N 33/68

(52) U.S. Cl. CPC G01N 33/6818

(57) ABSTRACT

The technology described herein is directed to methods for obtaining partial sequence information from a target protein. Also described herein are systems, devices, and kits for obtaining partial sequence information from a target protein.

### CROSS-REFERENCE TO RELATED APPLICATIONS

This application claims benefit under 35 U.S.C. § 119(e) of U.S. Provisional Application No. 63/227,560 filed Jul. 30, 2021, the contents of which are incorporated herein by reference in their entirety.

### GOVERNMENT SUPPORT

This invention was made with Government support under GM140211 awarded by the National Institutes of Health. The Government has certain rights in the invention.

### TECHNICAL FIELD

The technology described herein relates to methods for obtaining sequence information from a target protein.

### BACKGROUND

Recent advances in high-throughput DNA sequencing have broadly transformed biological research and biomedicine, and led to single-cell sequencing and precision medicine. Compared to nucleic acids, proteins more directly reflect cellular states and dynamic changes, and are recognized as more effective biomarkers. Current mass spectrometry-based proteomics suffers from limited detection sensitivity (requiring 10⁵-10⁶ peptide molecules), and does not allow effective detection of low-abundance cellular proteins and biomarkers in small samples (e.g., single cells or liquid biopsy samples). Given that a PCR-like self-replication strategy for protein amplification is not within sight, there is an urgent need to develop an amplification-free (i.e. single-molecule) approach for accurate, unbiased protein identification and high-throughput profiling.

### SUMMARY

The technology described herein is directed to methods for obtaining partial sequence information from a target protein. Also described herein are systems, devices, and kits for obtaining partial sequence information from a target protein.

In one aspect, described herein is a method for obtaining partial sequence information from a target protein, comprising: (a) denaturing a protein; (b) labeling occurrences of one or more particular amino acids in the protein; (c) capturing the protein on a substrate via its N-terminus or C-terminus; (d) elongating the protein; and (e) imaging the substrate to detect labeled amino acids, thereby locating the particular amino acids in the protein, whereby partial sequence information is obtained for the target protein.

In some embodiments of any of the aspects, labeling occurrences of one or more particular amino acids comprised fluorescent labeling.

In another aspect, described herein is a method for obtaining partial sequence information from a target protein, comprising: (a) denaturing a protein; (b) attaching docking strands to particular amino acids in the protein; (c) capturing the protein on a substrate via its N-terminus or C-terminus; (d) elongating the protein; (e) repeatedly contacting the captured protein with fluorescently-labeled imager strands that transiently bind to respective docking strands attached to particular amino acids in the protein; and (f) imaging the substrate, thereby locating the particular amino acids in the protein, whereby partial sequence information is obtained for the target protein.

In some embodiments of any of the aspects, the docking strands and imager strands comprise nucleic acid strands.

In some embodiments of any of the aspects, the step of capturing the N-terminus of the protein of the substrate comprises contacting the N-terminus of the protein with a cross-linking agent comprising 2-Pyridinecarboxaldehyde (2PCA).

In some embodiments of any of the aspects, a cross-linking agent is Tetrazine-2-Pyridinecarboxaldehyde (TZ-2PCA).

In some embodiments of any of the aspects, the cross-linking agent specifically reacts with a moiety on the substrate.

In some embodiments of any of the aspects, the moiety on the substrate comprises trans-cyclooctene (TCO).

In some embodiments of any of the aspects, the step of capturing the C-terminus of the protein of the substrate comprises contacting the C-terminus of the protein with a cross-linking agent comprising oxazolone.

In some embodiments of any of the aspects, the step of elongating the protein comprises microfluidic elongation in a microfluidic device.

In some embodiments of any of the aspects, a microfluidic channel of the microfluidic device is at least 10 μm in width.

In some embodiments of any of the aspects, the microfluidic elongation comprises flowing fluid past the protein at a flow rate of at least 20 uL/min.

In some embodiments of any of the aspects, the fluid has a viscosity of at least 1.4 Pa·s.

In some embodiments of any of the aspects, the fluid comprises glycerol.

In some embodiments of any of the aspects, the fluid comprises a denaturant.

In some embodiments of any of the aspects, the denaturant is selected from the group consisting of urea, guanidine, and sodium dodecyl sulfate (SDS).

In some embodiments of any of the aspects, the step of elongating the protein comprises: (a) linking the N-terminus of the protein to a first substrate, and linking the C-terminus of the protein to a second substrate; or (b) linking the C-terminus of the protein to a first substrate, and linking the N-terminus of the protein to a second substrate.

In some embodiments of any of the aspects, the first substrate comprises a surface in a microfluidic device.

In some embodiments of any of the aspects, the second substrate is a microbead.

In some embodiments of any of the aspects, the method further comprises applying a fluid flow force, centrifugal force, or magnetic force to the second substrate.

In some embodiments of any of the aspects, the protein is elongated to at least 80% of its expected contour length.

In some embodiments of any of the aspects, the method further comprises the step of: determining a score for an observed pattern of amino acid labeling compared to an expected pattern of amino acid labeling.

In some embodiments of any of the aspects, partial sequence of the protein is determined if the score is above a pre-determined threshold.

In another aspect, described herein is a system comprising: (a) a substrate; (b) a protein cross-linked to the substrate via its N-terminus or C-terminus; (c) docking strands attached to particular amino acids in the protein; and (d) fluorescently-labeled imager strands that transiently bind to docking strands attached to particular amino acids in the protein.

In another aspect, described herein is a microfluidic device comprising: (a) a cross-linking reagent; (b) docking strands attached to particular amino acids in a protein; (c) fluorescently-labeled imager strands that transiently bind to docking strands attached to particular amino acids in a protein; and (d) a high-viscosity and/or denaturing buffer.

In another aspect, described herein is a kit comprising: (a) a substrate; (b) a cross-linking reagent that permits attachment of a protein to the substrate; (c) docking strands comprising a functional group permitting attachment to particular amino acids in a protein; (d) fluorescently-labeled imager strands that transiently bind to respective docking strands; and (e) a high-viscosity and/or denaturing buffer.

In some embodiments of any of the aspects, such as a system as described herein, a microfluidic device as described herein, or a kit as described herein, the docking strands and imaging strands comprise nucleic acid strands.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-1C shows that DNA-PAINT super-resolution microscopy allows sensitive, accurate molecular detection. (FIG. 1A) DNA-PAINT uses transient binding between imager and docking strand to convert molecular information to blinking signal. (FIG. 1B) Frequency-based quantitative imaging (qPAINT) allows high accuracy (<5% error) molecular counting. (FIG. 1C) Discrete molecular imaging (DMI) allows high resolution (<5 nm) imaging in a dense molecular cluster.

FIG. 2A-2B shows that amino acid signatures of nucleocapsid (N) protein correctly distinguish SARS-CoV-2 virus and suggest its phylogenetic origin. (FIG. 2A) Accurate amino acid counting for three amino acids (K, M, Y) identified 2019-nCoV as a novel coronavirus. (FIG. 2B) Amino acid linear signatures further reveal their genetic similarities and suggest its close relationship to bat coronavirus RaTG13. Source: GenBank (SARS-CoV-2, SARS, MERS), GISAID (bat RaTG13).

FIG. 3A-3D shows that micro-bead based centrifugal stretching allows greater than 50 pN extension force. (FIG. 3A-3B) Schematic of centrifugal force microscope (CFM) setup, and micro-beads attached to DNA samples. (FIG. 3C) Image of high-throughput micro-bead pulling experiments. (FIG. 3D) DNA overstretching transition measured with CFM.

FIG. 4A-4B shows strategies for single-cell lysis and optical isolation on microfluidic device. (FIG. 4A) Left, schematic for protein capture after bacterial cell lysis. Right, single-cell pulldown with antibody detection. (FIG. 4B) Left, schematic for bacterial single-cell isolation following time-lapse imaging (SIFT) device. Right, image of a bacterial cell being trapped and moved with an optical trap.

FIG. 5A-5C shows a schematic of amino acid-specific protein-DNA labelling (Subsection 1.1). (FIG. 5A) Schematic for high-density DNA strand labelling on specific amino acids, in intact proteins. (FIG. 5B) Schematic for click chemistry-mediated two-step labelling approach. (FIG. 5C) Candidate crosslinkers for specific labelling of six amino acids (or a.a. class).

FIG. 6A-6B shows high-efficiency protein-DNA labelling on lysine (Subsection 1.1). (FIG. 6A) Gel electrophoresis shows high-efficiency lysine labelling with oligo on CYC (19 lysine, 1 N-term.). Three samples of increasing oligo concentrations are shown. The right-most lane shows complete labelling (20+/−1 oligos, 5% variation). (FIG. 6B) A panel of five model proteins all show high labelling efficiency on lysine.

FIG. 7A-7C shows a schematic for accurate molecular counting by DNA-PAINT and protein identification (Subsection 1.2). (FIG. 7A) DNA-PAINT converts molecular counts into repetitive blinking signal and allows for accurate molecular counting. (FIG. 7B) Exchange-PAINT allows counting of different DNA labels. (FIG. 7C) Schematic for amino acid counting based protein identification, illustrated in three dimensions for three amino acid counts.

FIG. 8 shows that DNA-PAINT allows accurate amino acid counting on single proteins (Subsection 1.2). Five model proteins were labelled with DNA oligos on lysine residues and assayed by DNA-PAINT on surface. Blinking kinetics measurement shows linear relationship between observed blinking kinetics and lysine count, with an average deviation of 1.8 only.

FIG. 9A-9D shows schematics for protein backbone extension and linear barcoding-based protein identification (Subsection 1.3). (FIG. 9A) Schematic for flow-based extension for proteins with dense DNA labels, and subsequent surface anchoring. (FIG. 9B) Schematic for micro-bead-based extension. (FIG. 9C) Schematic for high-resolution DNA-PAINT imaging with multiple amino acid labels after backbone extension. (FIG. 9D) Schematic for proteome library match of amino acid linear signature.

FIG. 10A-10C shows data for flow-based protein backbone extension (Subsection 1.3). Proteins were labelled with DNA and dyes, anchored on glass surface and stretched under flow. (FIG. 10A) Schematics and two single molecule (thyroglobulin, TG) examples. Three images before, during and after flow are shown, confirming the molecule has not been damaged or moved by the flow. (FIG. 10B-10C) A single molecule (apolipoprotein B-100) under increasing flow shows force-extension behavior consistent with worm-like chain (WLC) model. In both cases, extension to >90% of estimated contour length was observed.

FIG. 11A-11B shows a schematic for surface-based protein capture and serial amino acid specific labelling (Subsection 2.1). (FIG. 11A) Schematic for surface capture of protein mixture samples by pre-treatment with protein termini-specific crosslinker. (FIG. 11B) Schematic for serial DNA labelling of multiple amino acids. Unreacted amino acid or crosslinkers are capped after each round to prevent off-target labelling.

FIG. 12A-12B shows a schematic for microfluidic single-cell lysis and protein capture (Subsection 2.2). (FIG. 12A) Schematic for enzymatic lysis and surface protein capture for single bacterial cells. Cells were pre-diluted to low surface density to allow separation collection of proteins from different cells. (FIG. 12B) Schematic for micro-well cell trapping, lysis and protein capture from single mammalian cells, inside micro-wells.

FIG. 13 is an image showing exemplary protein labeling and stretching.

FIG. 14 is a schematic showing an exemplary bioinformatic analysis.

### DETAILED DESCRIPTION

Described herein is a technology that is capable of accurate, high-throughput protein identification from unknown samples at the single-molecule level. The premise of this research is that super-resolution microscopy can sensitively extract amino acid signatures (e.g., their abundances, or linear distribution along the protein's primary sequence) from single, intact protein molecules, which provide accurate identification and high throughput for protein profiling. This technology combines, for example, high-sensitivity, high-resolution DNA-PAINT imaging, high-efficiency protein labelling, protein backbone extension, and microfluidic control for single-cell manipulation. Specifically, described herein are: (1) biochemistry, microscopy, biophysics, and computational methods that permit high-throughput, single-molecule protein identification using specific amino acid signatures; and (2) a microfluidic workflow comprising single-cell lysis, protein capture and modification, and single-molecule imaging that permits single-cell proteomics. It is contemplated herein that such methods can be used for high-throughput, in-depth proteomic studies in a wide range of basic research and clinical contexts, including single-cell proteomics (e.g., for mammalian and bacterial samples), discovery of low-abundance biomarkers, and identification of new pathogens. Furthermore, concepts and methods described herein (e.g. high-efficiency protein-DNA labelling, protein backbone extension) can form the basis of biophysical studies and biotechnological developments.

Described herein are methods of single-molecule protein identification via elongation or stretching, which includes at least one of the following features: (1) a surface anchoring method that is compatible with denaturants and stable under high force; (2) a stretching method (e.g., using a microfluidic setup) that allows high stretching force, e.g., to extend the protein backbone, e.g., to as much as 90% or greater of its expected contour length; (3) a method that allows anchoring of the stretched protein on a surface and subsequent super-resolution imaging; and/or (4) a computational analysis for proteome coverage and identification. For background, see e.g., U.S. Pat. No. 10,006,917, the content of which is incorporated herein by reference in its entirety.

There are a variety of existing proteomic technologies including Edman degradation, mass spectrometry, and targeted methods (e.g., immuno-assays, microscopy); the single-molecule protein ID methods described herein have higher proteome coverage, detection sensitivity, and dynamic range compared to these other proteomic technologies. As used herein, the term “proteome coverage” refers to the number of different proteins that the method can detect. As used herein, the term “detection sensitivity” refers to the lowest number of molecules of a protein that the method can detect above background. As used herein, the term “dynamic range” refers to the ratio between the largest and smallest values (e.g., protein concentration or protein quantity) that the method can detect. Edman degradation has no proteome coverage or dynamic range since it can only analyze a single purified protein; its detection sensitivity is also low (e.g., >10¹² molecules). While mass spectrometry has high proteome coverage that is close to full coverage, it only has medium detection sensitivity (e.g., >10⁶ molecules) and low dynamic range (e.g., <10³). While targeted methods (e.g., immuno-assays, microscopy) have high detection sensitivity (e.g., down to single molecules), it only has low to medium proteome coverage (e.g., 100-1000 proteins) and medium dynamic range (e.g., ˜10³-10⁶). In contrast, the single-molecule protein ID methods described herein have high proteome coverage that is close to or at full coverage, high detection sensitivity (e.g., down to single molecules), and high dynamic range (e.g., >10⁶).

The single-molecule protein ID methods described herein also exhibit benefits over sequencing technologies such as proteasome-based sequencing, Erdman degradation based sequencing, and N-terminal based sequencing. While proteasome-based sequencing has a fast readout, it detects inaccurate distances and is subject to photobleaching. While Edman degradation can exhibit single amino acid resolution, it is subject to short read length and photobleaching. While N-terminal based sequencing has an N-terminal specific signal, it exhibits limited cutter positions and efficiency. In contrast, the single-molecule protein ID methods described herein have long read length, accurate distance detection, and no photobleaching. See e.g., Ginkel et al., PNAS Mar. 27, 2018 115 (13) 3338-3343; Swaminathan et al., Nature Biotechnology volume 36, pages 1076-1082 (2018); quantum-si.com available on the world wide web; the contents of each of which are incorporated herein by reference in their entireties.

In one aspect, the method comprises the following sequential steps: (a) sample preparation and protein extraction; (b) residue labeling and surface fixation; (3) protein stretching; (4) multiplexed, Discrete Molecular Imaging (DMI) super-resolution imaging; and (5) computation identification. Such a method allows for at least the following applications: direct microscopy readout (e.g., super-resolution); affinity based reagents; pairwise distance measurement; cutter-based measurement; and/or in-situ based readout.

Described herein is a surface anchoring method, e.g., specific for the N terminus or C terminus of proteins. In some embodiments, a cross-linking agent is used that reacts with the N terminus of a protein and a moiety on a substrate. In some embodiments, a cross-linking agent is used that reacts with the C terminus of a protein and a moiety on a substrate. Examples of substrates include, but are not limited to, microfluidic devices, microparticles or microbeads, nanotubes, microtiter plates, medical apparatuses (e.g., needles or catheters) or implants, dipsticks or test strips, microchips, filtration devices or membranes, diagnostic strips, hollow-fiber reactors and other solid substrates, as well as nucleic acid scaffolds, protein scaffolds, lipid scaffolds, dendrimers, living cells and biological tissues or organs, extracorporeal devices, and mixing elements (e.g., spiral mixers).

In some embodiments, the cross-linking agent that reacts with the N terminus of a protein comprises 2PCA (2-Pyridinecarboxaldehyde); see e.g., MacDonald et al. 2015, Nature chemical biology 11 (5):326-31, the content of which is incorporated herein by reference in its entirety. In some embodiments, the cross-linking agent that reacts with the N terminus of a protein is TZ-2PCA (e.g., Tetrazine-2-Pyridinecarboxaldehyde; see e.g., Formula I below). The TZ (e.g., tetrazine) end of TZ-PCA reacts with a moiety (e.g., TCO) on the substrate. The 2PCA (e.g., 2-Pyridinecarboxaldehyde) end of TZ-PCA reacts with the N terminus of a protein. In some embodiments, the moiety on the substrate is TCO (e.g., trans-cyclooctene; see e.g., Formula II below). Such a method results in covalent and N-terminal specific protein-surface linkage ( ). In some embodiments, the protein is pre-treated with a N-deblocking aminopeptidase (e.g., Pyrococcus furiosus (Pfu) N-acetyl Deblocking Aminopeptidase (Ac-DAP); e.g., TAKARA Cat. 7340).

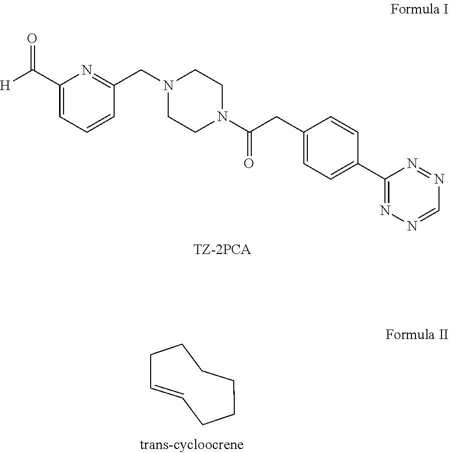 [SMILES: O=Cc1cccc(CN2CCN(C(=O)Cc3ccc(-c4nncnn4)cc3)CC2)n1 ; C1=C/CCCCCC/1]

In some embodiments, the cross-linking agent that reacts with the C terminus of a protein comprises oxazolone (see e.g., Formula III, below). See e.g., Yamaguchi et al., 2006, Analytical Chemistry 78 (22):7861-7869, the content of which is incorporated herein by reference in its entirety.

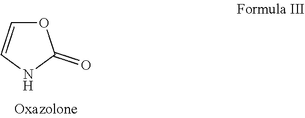

Described herein is a method of elongating a protein, e.g., using microfluidic stretching. In some embodiments, a microfluidic channel of, e.g., 10 μm in width is used to flow fluid towards a substrate to which a protein is attached. In some embodiments, the microfluidic channel has a width of at least 1 μm, at least 2 μm, at least 3 μm, at least 4 μm, at least 5 μm, at least 6 μm, at least 7 μm, at least 8 μm, at least 9 μm, at least 10 μm, at least 11 μm, at least 12 μm, at least 13 μm, at least 14 μm, at least 15 μm, at least 16 μm, at least 17 μm, at least 18 μm, at least 19 μm, at least 20 μm, at least 30 μm, at least 40 μm, or at least 50 μm, or more. In some embodiments, the microfluidic channel has a width of at most 1 μm, at most 2 μm, at most 3 μm, at most 4 μm, at most 5 μm, at most 6 μm, at most 7 μm, at most 8 μm, at most 9 μm, at most 10 μm, at most 11 μm, at most 12 μm, at most 13 μm, at most 14 μm, at most 15 μm, at most 16 μm, at most 17 μm, at most 18 μm, at most 19 μm, at most 20 μm, at most 30 μm, at most 40 μm, or at most 50 μm.

In some embodiments, the flow rate of the fluid in the microfluidic channel is at least 20 μL/min. In some embodiments, the flow rate of the fluid in the microfluidic channel is at least 1 μL/min, at least 2 μL/min, at least 3 μL/min, at least 4 μL/min, at least 5 μL/min, at least 6 μL/min, at least 7 μL/min, at least 8 μL/min, at least 9 μL/min, at least 10 μL/min, at least 11 μL/min, at least 12 μL/min, at least 13 μL/min, at least 14 μL/min, at least 15 μL/min, at least 16 μL/min, at least 17 μL/min, at least 18 μL/min, at least 19 μL/min, at least 20 μL/min, at least 30 μL/min, at least 40 μL/min, at least 50 μL/min, at least 60 μL/min, at least 70 μL/min, at least 80 μL/min, at least 90 μL/min, at least 100 μL/min, at least 110 μL/min, at least 120 μL/min, at least 130 μL/min, at least 140 μL/min, at least 150 μL/min, at least 160 μL/min, at least 170 μL/min, at least 180 μL/min, at least 190 μL/min, at least 200 μL/min or more.

In some embodiments, the fluid in the microfluidic channel is high viscosity. In some embodiments, the viscosity of the fluid in the microfluidic channel is at least 1.412 Pa·s (1 Pa·s is equivalent to 1 newton-second per square meter). In some embodiments, the viscosity of the fluid in the microfluidic channel is at least 0.5 Pa·s, at least 1.0 Pa·s, at least 1.1 Pa·s, at least 1.2 Pa·s, at least 1.3 Pa·s, at least 1.4 Pa·s, at least 1.5 Pa·s, at least 1.6 Pa·s, at least 1.7 Pa·s, at least 1.8 Pa·s, at least 1.9 Pa·s, or at least 2.0 Pa·s, or more. In some embodiments, the fluid in the microfluidic channel comprises glycerol.

In some embodiments, the fluid in the microfluidic channel comprises a denaturing buffer. In some embodiments, the denaturant (i.e., in a denaturing buffer) is selected from the group consisting of urea, guanidine, detergent (e.g., sodium dodecyl sulfate (SDS), Triton X-100), organic solvents (e.g., ethanol), acids and bases (e.g., sodium bicarbonate, acetic acid). In some embodiments, the denaturant is selected from the group consisting of urea, guanidine, and SDS. In some embodiments, the denaturant is SDS.

In some embodiments, the observed length of the protein increases as the flow rate of the fluid in the microfluidic channel increases. In some embodiments, the protein is elongated (e.g., using the elongation methods as described herein; e.g., microfluidics, surface stretching, etc.) to at least 90% of its expected contour length. In some embodiments, the protein is elongated to at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or 100% of its expected contour length.

Described herein is a method of elongating a protein, e.g., using surface stretching (e.g., substrate stretching, substrate elongation). In some embodiments, the N-terminus of the protein is linked to a first substrate, and the C-terminus of the protein is linked to a second substrate. In some embodiments, the C-terminus of the protein is linked to a first substrate, and the N-terminus of the protein is linked to a second substrate. In some embodiments, the first substrate is a microfluidic device (e.g., chamber, lumen or well of a microfluidic device). In some embodiments, the second substrate is a microbead (e.g., polymer microbeads, magnetic microbeads, and the like). In some embodiments, the protein linked is stretched by applying a fluid flow force, centrifugal force, or magnetic force to the second substrate.

Described herein is a method of locating the position of particular amino acids in a protein; such a method can comprise a bioinformatic analysis for amino acid pattern matching. In some embodiments, the bioinformatic method comprises using Formula IV below, e.g., to generate a score for the observed pattern of amino acid labeling compared to an expected pattern of amino acid labeling. In some embodiments, the partial sequence of the protein is determined if the score is above a pre-determined threshold.

$$score = λ … \sum ( {e}^{- \frac{1}{2} {( \frac{{x}_{ref} - {x}_{ref \to {obs}_{b}}}{{σ}_{res}} )}^{2} - \frac{1}{2} {( \frac{b}{{σ}_{res}} )}^{2}} ) + {λ}_{+} \sum ( {e}^{- \frac{1}{2} {( \frac{{x}_{ref} - {x}_{ref \to {obs}_{b}}}{{σ}_{res}} )}^{2} - \frac{1}{2} {( \frac{b}{{σ}_{res}} )}^{2}} ) \qquad Formula IV$$

Features of this bioinformatic method include at least one of the following: (1) bidirectional mapping between reference and observed patterns; (2) separate terms for missing labels and mis-labelling error; (3) imaging resolution can be adapted to a local pattern; (4) score-based matching, allowing false discovery rate (FDR) analysis; and/or (5) the method allows for un-stretched region(s) within the protein.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA.

As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

As used herein, “contacting” refers to any suitable means for delivering, or exposing, an agent to at least one protein, cell or moiety. Exemplary delivery methods include, but are not limited to, direct delivery to a protein preparation, biological sample, cell, cell preparation or cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A method for obtaining partial sequence information from a target protein, comprising
   a) denaturing a protein;
   b) labeling occurrences of one or more particular amino acids in the protein;
   c) capturing the protein on a substrate via its N-terminus or C-terminus;
   d) elongating the protein; and
   e) imaging the substrate to detect labeled amino acids, thereby locating the particular amino acids in the protein, whereby partial sequence information is obtained for the target protein.
2. The method of paragraph 1, wherein labeling occurrences of one or more particular amino acids comprised fluorescent labeling.
3. A method for obtaining partial sequence information from a target protein, comprising
   a) denaturing a protein;
   b) attaching docking strands to particular amino acids in the protein;
   c) capturing the protein on a substrate via its N-terminus or C-terminus;
   d) elongating the protein;
   e) repeatedly contacting the captured protein with fluorescently-labeled imager strands that transiently bind to respective docking strands attached to particular amino acids in the protein; and
   f) imaging the substrate, thereby locating the particular amino acids in the protein, whereby partial sequence information is obtained for the target protein.
4. The method of paragraph 3, wherein the docking strands and imager strands comprise nucleic acid strands.
5. The method of any one of paragraphs 1-4, wherein the step of capturing the N-terminus of the protein of the substrate comprises contacting the N-terminus of the protein with a cross-linking agent comprising 2-Pyridinecarboxaldehyde (2PCA).
6. The method of paragraph 5, wherein a cross-linking agent is Tetrazine-2-Pyridinecarboxaldehyde (TZ-2PCA).
7. The method of paragraph 5 or 6, wherein the cross-linking agent specifically reacts with a moiety on the substrate.
8. The method of paragraph 7, wherein the moiety on the substrate comprises trans-cyclooctene (TCO).
9. The method of any one of paragraphs 1-4, wherein the step of capturing the C-terminus of the protein of the substrate comprises contacting the C-terminus of the protein with a cross-linking agent comprising oxazolone.
10. The method of any one of paragraphs 1-9, wherein the step of elongating the protein comprises microfluidic elongation in a microfluidic device.
11. The method of paragraph 10, wherein a microfluidic channel of the microfluidic device is at least 10 μm in width.
12. The method of paragraph 10 or 11, wherein the microfluidic elongation comprises flowing fluid past the protein at a flow rate of at least 20 uL/min.
13. The method of any one of paragraphs 10-12, wherein the fluid has a viscosity of at least 1.4 Pa·s.
14. The method of any one of paragraphs 10-13, wherein the fluid comprises glycerol.
15. The method of any one of paragraphs 10-14, wherein the fluid comprises a denaturant.
16. The method of paragraph 15, wherein the denaturant is selected from the group consisting of urea, guanidine, and sodium dodecyl sulfate (SDS).
17. The method of any one of paragraphs 1-4, wherein the step of elongating the protein comprises:
   a) linking the N-terminus of the protein to a first substrate, and linking the C-terminus of the protein to a second substrate; or
   b) linking the C-terminus of the protein to a first substrate, and linking the N-terminus of the protein to a second substrate.
18. The method of paragraph 17, wherein the first substrate comprises a surface in a microfluidic device.
19. The method of paragraph 17 or 18, wherein the second substrate is a microbead.
20. The method of any one of paragraphs 17-19, further comprising applying a fluid flow force, centrifugal force, or magnetic force to the second substrate.
21. The method of any one of paragraphs 1-20, wherein the protein is elongated to at least 80% of its expected contour length.
22. The method of any one of paragraphs 1-21, further comprising the step of: determining a score for an observed pattern of amino acid labeling compared to an expected pattern of amino acid labeling.
23. The method of paragraph 22, wherein partial sequence of the protein is determined if the score is above a pre-determined threshold.
24. A system comprising:
   a) a substrate;
   b) a protein cross-linked to the substrate via its N-terminus or C-terminus;
   c) docking strands attached to particular amino acids in the protein; and
   d) fluorescently-labeled imager strands that transiently bind to docking strands attached to particular amino acids in the protein.
25. A microfluidic device comprising:
   a) a cross-linking reagent;
   b) docking strands attached to particular amino acids in a protein;
   c) fluorescently-labeled imager strands that transiently bind to docking strands attached to particular amino acids in a protein; and
   d) a high-viscosity and/or denaturing buffer.
26. A kit comprising:
   a) a substrate;
   b) a cross-linking reagent that permits attachment of a protein to the substrate;
   c) docking strands comprising a functional group permitting attachment to particular amino acids in a protein;
   d) fluorescently-labeled imager strands that transiently bind to respective docking strands; and
   e) a high-viscosity and/or denaturing buffer.
27. The system of paragraph 24, microfluidic device of paragraph 25 or kit of paragraph 26, wherein the docking strands and imaging strands comprise nucleic acid strands.

The technology described herein is further illustrated by the following examples which in no way should be construed as being further limiting.

### EXAMPLES

### Example 1: Single-Molecule Protein Identification and Single-Cell Proteomics

Recent advances in high-throughput DNA sequencing has broadly transformed biological research and biomedicine, and led to single-cell sequencing and precision medicine. Compared to nucleic acids, proteins more directly reflect cellular states and dynamic changes, and are recognized as more effective biomarkers. A high-throughput, unbiased protein profiling method will permit proteomic studies in small samples (e.g. single cells, liquid biopsy samples) and detection of low-abundance biomarkers, and equally broadly transform the practice in many fields, including cancer, immunology, aging and neurodegeneration. Mass spectrometry is a powerful tool for unbiased proteomics. However, it suffers from limited detection sensitivity (currently requires 10⁵-10⁶ molecules). Since half of the human proteome is estimated to be expressed at <50,000 copies per cell, this limited detection sensitivity prevents mass spectrometry from effectively detecting low-abundance proteins and biomarkers in single cells or liquid biopsy samples. Although targeted detection methods have achieved much lower detection limit (10³˜10⁴ molecules), they require the use of high-affinity and high-specificity antibody pairs that are not currently available for many cellular proteins, and are further limited in multiplexing capacity due to unspecific binding and cross-talk (currently <100 targets). Given that a PCR-like self-replication strategy for protein amplification is not within sight, the current urgent need, is to develop a robust amplification-free (i.e. single-molecule) approach for unbiased protein identification and high-throughput profiling.

It is a goal to develop single-molecule and high-throughput protein profiling methods, and apply them for in-depth single-cell proteomic studies in mammalian and bacterial samples, and the detection of low-abundance biomarkers in bodily fluids. A step towards this goal, is to develop the basic technological platform that (i) permits protein identification at single-molecule level and with high throughput by amino acid-specific labelling and imaging, and (ii) implements single-cell proteomics using microfluidics-based protein profiling methods. A central principle is that super-resolution microscopy can sensitively extract amino acid signatures from single, intact protein molecules, which provide accurate identification and high throughput for protein profiling. Exemplary bioinformatics analysis shows that accurate detection of (i) amino acid abundance, or (ii) their sequence distribution, allows for robust protein identification from the human proteome. In one aspect, provided herein is an adaptation of a high-sensitivity, super-resolution optical detection methods (DNA-PAINT) that was previously developed, for accurate molecular counting (<5% error) and imaging with molecular resolution (<5 nm).

1. A method for high-throughput, single-molecule protein identification using specific amino acid signatures. In this section, described herein is the development of an experimental workflow that realizes this strategy, comprising three steps: (i) high-efficiency protein labelling and surface immobilization, (ii) high-sensitivity and high-resolution single-molecule imaging with optional protein backbone extension, and (iii) computational analysis and protein identification. Specifically, biochemistry methods are developed that allow high-efficiency, specific amino acid labelling with DNA barcodes (Subsection 1.1), DNA-PAINT based high-sensitivity and high-resolution single-molecule microscopy methods for faithful readout of the amino acid signatures, optionally with hydrodynamic or mechanic protein backbone extension (Subsection 1.2, and 1.3,), and data analysis platform for robust assignment of protein identity from imaging results (Subsection 1.4). These methods are applied first for model proteins, then in complex mixture samples (human cell lysate and organelle-specific lysate, e.g. mitochondria).

2. A microfluidic workflow for single-molecule protein identification and single-cell proteomics. In this section, described herein is the development of a microfluidic workflow for integrated single-cell lysis, protein capture and modification, and single-molecule imaging. Specifically, a surface protein capture and specific amino acid labelling method is developed for miniaturized sample preparation (Subsection 2.1), and an integrated microfluidic workflow for on-chip cell lysis, protein capture and single-molecule proteomic profiling, adapting from previous microfluidic cell culture, single-cell isolation and lysis methods (Subsection 2.2).

Outcomes include a technology platform for accurate, unbiased protein identification at single molecules and high-throughput profiling for in-depth proteomic studies in a wide range of basic research and clinical contexts. This research is expected to have a broad impact in biological research and biomedicine, in particular allowing single-cell proteomics (for mammalian and bacterial samples), discovery of low-abundance disease biomarkers, and identification of new pathogens (e.g. SARS-CoV-2). Equally important, many innovative techniques and methods developed during this research (e.g. high-efficiency protein-DNA labelling, protein backbone extension) will form the basis and permit new detection and analysis regimes for future biophysical studies and biotechnological developments.

The ability to both understand biological systems and to translate these understandings into effective diagnostics and therapies has more and more depended on sensitive and high-throughput analytical platforms. Next-generation sequencing (NGS) has enabled massively parallel identification of DNA and RNA molecules across different samples, notably in single-cell and cell-free samples, leading to new diagnostic tools and the development of new therapies. Compared to nucleic acids, proteins more directly reflect cellular states and dynamic changes, and are recognized as more effective biomarkers. A technology capable of generating robust single-molecule protein profiling data across various biological systems, with the high-sensitivity and coverage comparable to NGS, is critical in advancing the understanding of the cellular proteome and should offer an important new perspective of disease. Currently, proteomic studies mainly rely on digestion-based (“bottom-up”) mass spectrometry (MS) platforms. While entire proteomes have been mapped in bulk, fractionated samples, the attomole sensitivity of MS (i.e. 10⁵-10⁶) peptides (Lombard-Banek et al. 2016)) precludes in-depth proteomic profiling in single cells, as most cellular proteins exist well below this detection level. In fact, half of the human proteome is estimated to be expressed at <50,000 copies per cell (Schwanhausser et al. 2011), and many functionally important bacterial proteins are expressed at <100 copies per cell (Taniguchi et al. 2010). As a result, recently reported MS-bases single-cell proteomic analysis observed only 2,000 or fewer of the most abundant cellular proteins (Budnik et al. 2018). Given that a PCR-like self-replication strategy for protein amplification is not within sight, technologies for higher-sensitivity, single-molecule proteomics study are necessary to address this challenge.

Antibody probe-based multiplex detection methods (such as CyTOF, digital ELISA, PLA, PEA, SCBC (Bendall et al. 2011, Rissin et al. 2010, Albayrak et al. 2016, Assarsson et al. 2014, Shi et al. 2012)) offer an 100-fold improvement in sensitivity over MS (10³˜10⁴ molecules), but require the use of high-affinity and high-specificity antibody pairs that are currently not available for many cellular proteins, and are limited in multiplexing power due to unspecific binding and cross-reactivity (currently <100 targets). Furthermore, recent concerns have been raised about the poor quality and specificity of antibodies used to capture biomarkers (Marcon et al. 2015). The possibility for adapting Edman degradation to single-molecule peptide identification was recently reported. This involves specific labelling of amino acids with organic fluorophores prior to Edman degradation, and detection via total internal reflection fluorescence (TIRF) microscopy (Swaminathan et al. 2018). Like MS, Edman degradation is also limited to peptide fragments, and suffers from similar pre-processing bias. A similar approach with protease-based enzymatic degradation was also recently explored, via single-molecule FRET readout (Ginkel et al. 2018). Stochastic shifts in processing speed and backtracking of protease, and poor signal-to-noise ratio (SNR), are major hurdles for such methods. Nanopore-based approaches with either label-free detection or optical readout have also been proposed (Nivala et al. 2014, Ohayon et al. 2019), but suffers from similar non-uniform translocation speeds and poor SNR detection, and do not allow accurate single-molecule identification.

A robust and general method for high-throughput, single-molecule protein identification from an unknown, complex mixture remains elusive. The methods described herein establish a novel strategy for addressing this challenge, by high-sensitivity super-resolution imaging (DNA-PAINT) readout of specific amino acid signatures on single protein molecules, that allows robust protein identification of unknown identities. The methods described herein research will further establish a strategy for miniaturization in microfluidic devices and permit in-depth proteomics in single cell samples.

Specifically, this example will establish two complementary approaches for single-molecule protein identification based on two amino acid signature models, both with high throughput (10⁴-10⁵ molecules per DNA-PAINT imaging session). These approaches will permit high-sensitivity proteomic profiling in various research and clinical contexts, including in-depth single-cell proteomics, in situ imaging-based proteomics, proteomic microbiome profiling, biomarker detection in clinical samples, and potentially identification of new pathogens (e.g. infectious viruses). The methods described herein will further help translate research into new clinical applications by introducing new concepts and strategies, including bottlebrush protein-DNA hybrids and protein backbone extension methods. These could have broad impact in facilitating new approaches in molecular diagnostics and therapies.

Section 1: A Method for High-Throughput, Single-Molecule Protein Identification by Specific Amino Acid Signatures.

1. DNA-PAINT Super-Resolution Microscopy Method Allows Sensitive and Accurate Optical Detection of Single Molecules.

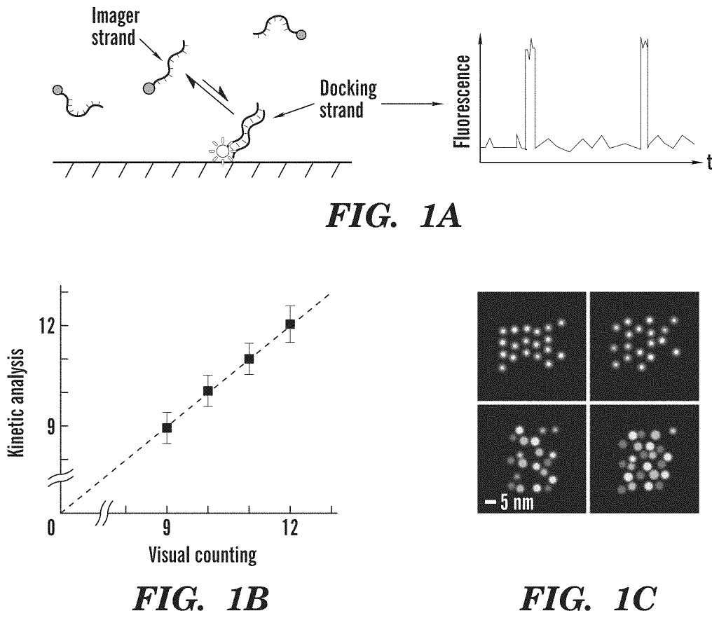

Super-resolution imaging using DNA-PAINT is performed by dynamic binding and unbinding of fluorophore-labelled short DNA “imager” strands onto a complementary “docking” strand labelled on the target sample [FIG. 1a]. Due to the repetitive binding of imager strands, DNA-PAINT is not limited by dye blinking or photobleaching, and allows ultra-sensitive detection of single-molecule targets (>97%, (Strauss et al. 2018, Dai, et al. 2016), as compared to typical efficiency of <75% for STORM and PALM (Thevathasan et al. 2019, Durisic et al. 2014) imaging). This high detection efficiency is critical for quantitative detection of dozens of amino acid residues in a single protein molecule (e.g. for 75% single-target detection efficiency, the probability or correctly observing 20 targets in a protein would be as low as (0.75)\hat{ }20=0.3%). In particular relevance to this research, DNA-PAINT exhibits three unique advantages: accurate molecular counting with quantitative PAINT (qPAINT, <5% counting error (Jungmann et al. 2016)) [FIG. 1b], ultra-high imaging resolution with discrete molecular imaging (DMI, <5 nm resolution (Dai, et al. 2016)) [FIG. 1c], and spectrally-unlimited, high multiplexing power with orthogonal imager strands (Exchange-PAINT, 10+“colors” (Jungmann et al. 2014)).

2. Accurate Detection of Amino Acid Signatures Allows Accurate Protein Identification in Single Molecules.

In this example, described herein are two models for protein-specific amino acid signatures: (i) abundance, (ii) positions along the protein's primary sequence. The analysis shows that, using previously demonstrated imaging accuracies and estimated experimental defects, both models allow accurate protein identification from complex mixture samples, such as human cellular and organelle proteomes. In particular, for model (i), labelling two specific amino acids (lysine and cysteine) allows 50-90% of proteins to be uniquely identified in specific subcellular compartments (much more favorable compared to (Swaminathan et al. 2018)). For model (ii), labelling with a single amino acid (e.g. lysine) already allows unique identification of 96% (error-free, 5 nm resolution, 2% FDR) of the entire human proteome (excluding short proteins), or 50% assuming 20% labelling error and 10 nm resolution.

Labelling with more specific amino acids, reducing imaging error, or increasing resolution will further significantly improve the coverage (e.g. close to 100% coverage for lysine+cysteine labelling without errors, 2% FDR, or 75% with 20% error) [Table 1]. Compared to previously proposed peptide-based amino acid signatures for Edman degradation (Swaminathan et al. 2015) and single-molecule FRET (Yao et al. 2015)), the models described here extract maximal information along the entire protein backbone, and allow accurate and robust protein identification from complex mixtures, as well as de novo identification capability, even with sparse amino acid labelling.

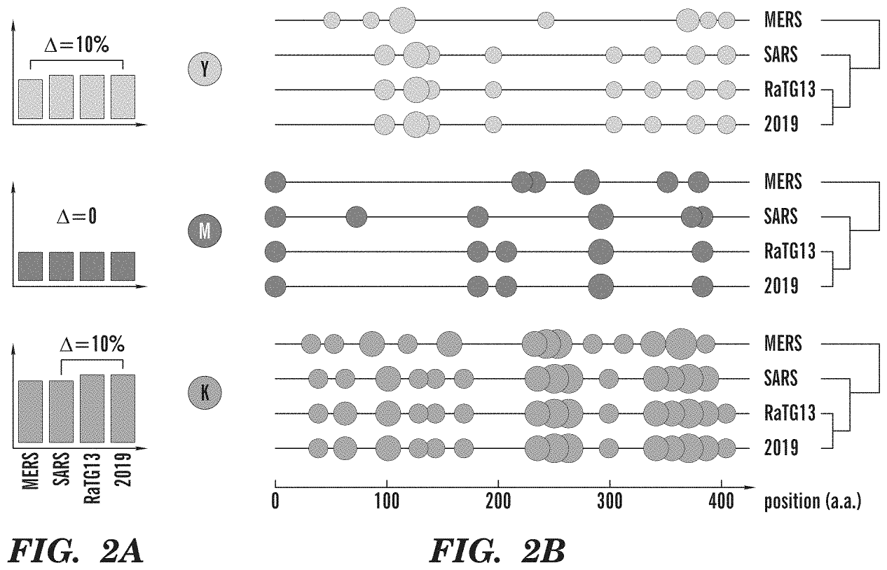

A particularly informative example to illustrate the identification power of this new method is to consider the SARS-CoV-2 coronavirus, which has recently caused a global pandemic with serious human health and economic consequences. As a complementary approach to nucleic acid based molecular testing, a sensitive, de novo (antibody-free) protein detection method could potentially provide an alternative for both clinical diagnostics and viral identification. FIG. 2 shows that, using either amino acid model, SARS-CoV-2 can be distinguished from the other two closely related coronavirus species (SARS and MERS) that also caused world-wide outbreaks previously. Furthermore, their linear amino acid signatures correctly reflect mutual genetic distances, suggesting phylogenetic origin (Zhou et al. 2020) [FIG. 2b].

3. Hydrodynamic Flow and Micro-Bead Pulling Allows Protein Backbone Extension to >80% of its Contour Length.

Protein backbone extension is critical for imaging the protein-specific amino acid patterns. It is fundamentally a combination of two processes: unfolding (kinetic) and straightening (thermodynamic). For unfolding, under physiological buffer and fast pulling conditions, mechanically stable protein domains unfold at 150-300 pN (Rief et al. 1997). Slow pulling, or longer incubation lowers the required pulling force logarithmically (down to <50 pN at 300 nm/hr, (Rief et al. 1997)), and the use of strong denaturing conditions significantly increases the unfolding rate (>100× at 6M GdmCl, (Guinn et al. 2015)). Upon successful unfolding, it has been reported 50 pN stretching force allows extension to 80% of its contour length (120 pN for 90% (Tskhovrebova et al. 1997)).

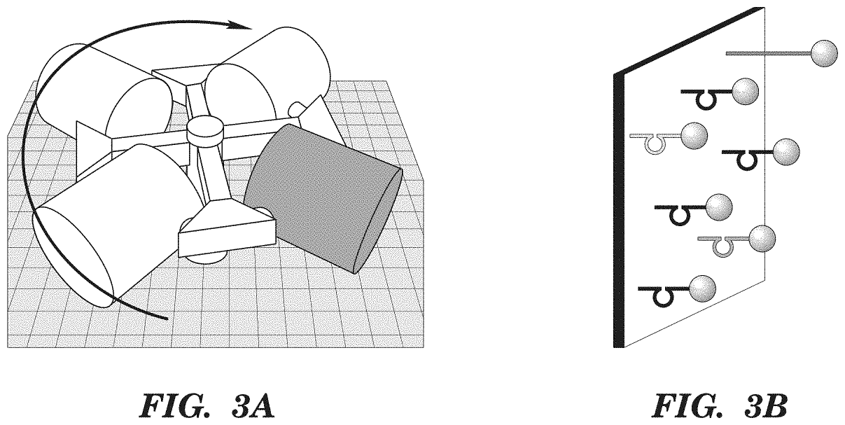

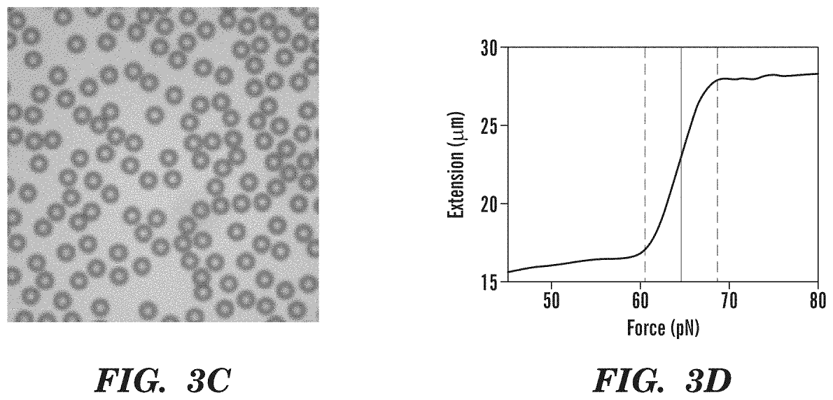

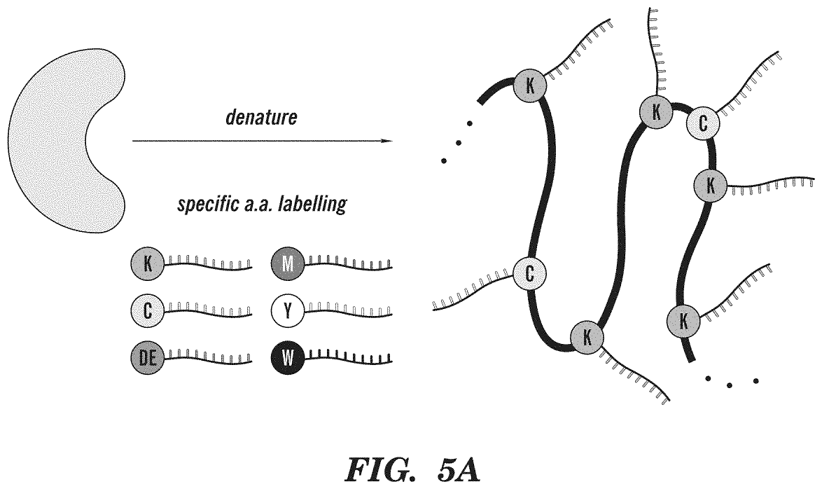

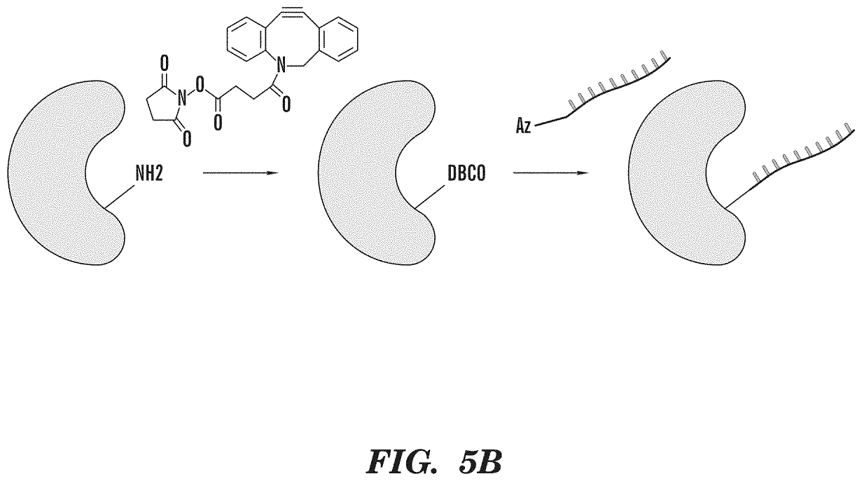

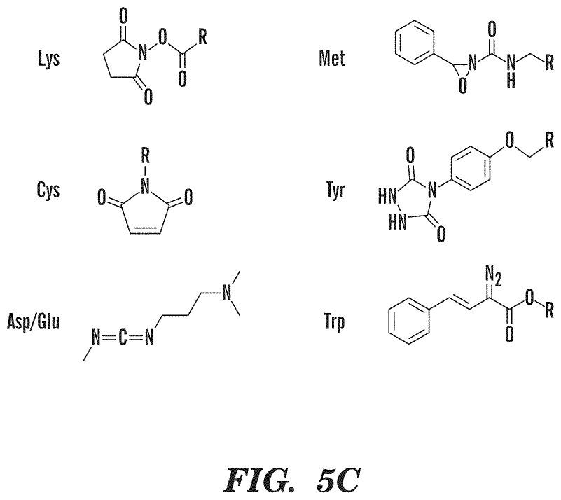

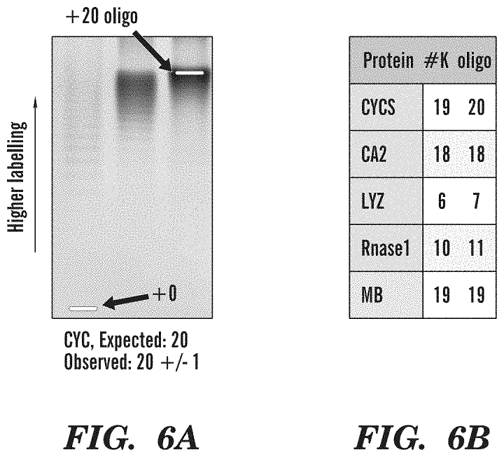

Herein are described two experimental implementations for protein backbone extension: (i) hydrodynamic flow for stretching protein samples with dense DNA labels, (ii) micro-bead based stretching by centrifugal force or flow. The analysis shows that, both methods provide enough pulling force (50 pN) for effective protein stretching. Specifically, for method (ii), centrifugal (Yang et al. 2016) [FIG. 3] or flow-based stretching methods developed can be used, which offer 50 pN pulling force. For method (i), it has been previously believed that hydrodynamic drag does not provide enough force for protein backbone extension. However, it is contemplated herein that with dense DNA labelling (forming a bottlebrush polymer), the collective drag experienced by all the DNA strands significantly increases the pulling force and allows effective protein stretching (Subsection 1.3, [FIG. 5]). Specifically, a simplified model using Stoke's law suggests that each DNA base (approximately 1 nm in diameter) generates 0.1 pN of pulling force under 10 mm/s aqueous flow, i.e. only 10 DNA strands of 50 bases each would generate enough pulling force for effective protein backbone extension. Increasing flow rate, use of high viscosity buffer, and higher degree of DNA labelling would all further increase the stretching force. The results (Subsection 1.3, [FIG. 6]) further support this conclusion.

Section 2: A Microfluidic Workflow for Single-Molecule Protein Identification and Single-Cell Proteomics.

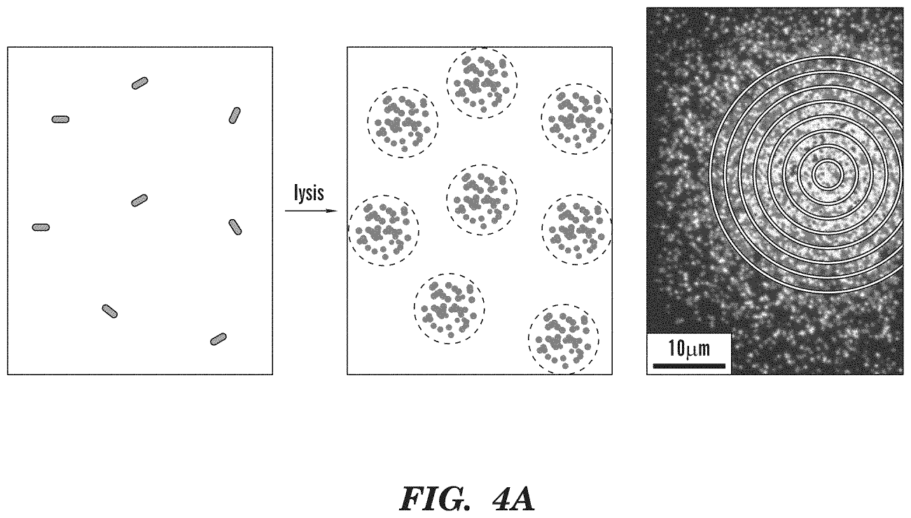

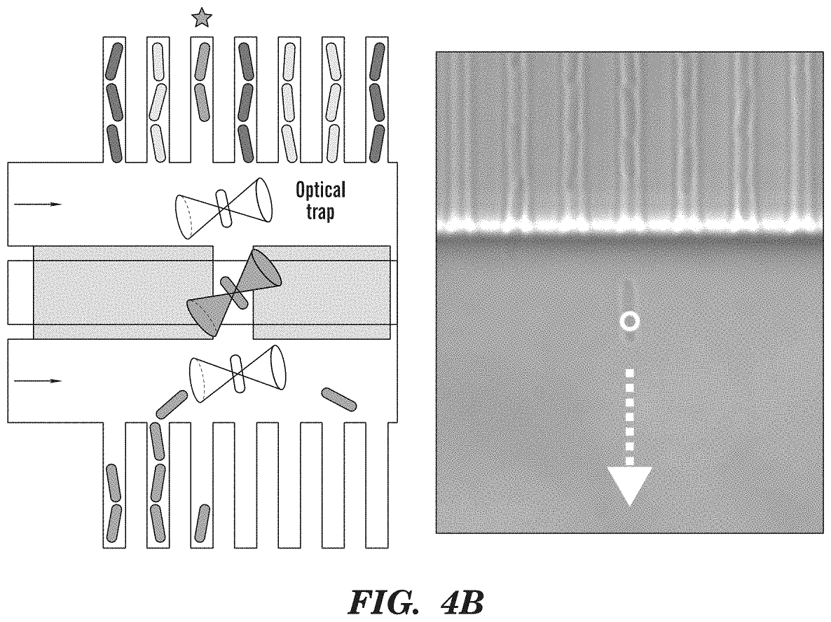

In this section, described herein is the development of two methods, for bacterial and mammalian single-cell proteomics, respectively. Microfluidics-based single-cell trapping, on-chip lysis and microscopy imaging methods for both bacterial and mammalian cells have been well-developed and reported previously (Prakadan et al. 2017, Potvin-Trottier et al. 2018). In particular, bacterial single-cell trapping and continuous growth in linear colonies (the “mother machine”) has been previous reported (Taheri-Araghi et al. 2015), on-chip enzymatic lysis and single-cell protein capture has been recently demonstrated (Wang et al. 2018) [FIG. 4a]. For mammalian single-cell analysis, micro-well based cell capture and lysis has been applied to targeted protein profiling in the form of microarray and miniature antibody array (Love et al. 2006, Shi et al. 2012). In particular, an integrated platform for bacterial single-cell isolation has been developed following linear colony and time-lapse imaging (SIFT) (Luro et al. 2020), as well as singe cell lysis on-chip, and this platform is being developed for human cells [FIG. 4b].

This research incorporates both conceptual and technical innovations. Conceptually, the following are introduced: new amino acid signatures for protein identification (their abundance, and position along the protein backbone), and new detection mechanisms for single-molecule readout (high-sensitivity super-resolution imaging on intact proteins with DNA labels).

Technically, the approach introduces several novel methods. Specifically, it develops new biochemical approaches for high-efficiency labelling of DNA strands onto specific amino acids, in high density and on intact proteins. Previous approaches for specific amino acid labelling on whole proteins only allowed high-efficiency labelling with small molecule tags, or DNA labelling on surface accessible amino acids, and the labelling efficiency was not precisely assessed. Also described herein is the development of new biophysical approaches for effective protein backbone extension, in a high-throughput and proteome-wide manner, without the requirement for genetically engineered attachment labels. Previous methods for successful protein backbone elongation (to >80% contour length) were based on atomic force microscope (AFM) or optical tweezer methods, that were low-throughput, and were typically performed on genetically engineered proteins, which is not compatible with unbiased proteomic studies, or with clinical samples.

Described herein is a strategy for high-throughput, single-molecule protein identification, that allows for comprehensive proteomics profiling in small and complex samples, such as single cells and liquid biopsy samples. The premise of this research is that, rather than recognizing the 3D structure and surface interactions, accurate single-molecule measurement of the abundance and/or distribution of specific amino acids within a protein's primary sequence also provides a unique, protein-specific signature, that allows for robust protein identification. Described herein is the development of an experimental workflow to realize such a strategy, using DNA barcodes to label specific amino acid, and high-sensitivity, single-molecule imaging (DNA-PAINT method) for accurate readout. In Section 1, described herein is the development of the biochemical, biophysical, microscopic and computational methods for implementing this workflow, comprising three steps: (i) high-efficiency amino acid labelling with DNA barcodes (Subsection 1.1), (ii) surface anchoring and DNA-PAINT single-molecule imaging with DNA-PAINT, optionally with protein backbone extension (Subsection 1.2 and 1.3), and (iii) computational analysis for protein identification and single-cell proteomic analysis (Subsection 1.4). In Section 2, described herein is the adaptation and miniaturization of this workflow into a microfluidic device and develop an integrated single-cell lysis and proteomics method.

Section 1. A Method for High-Throughput, Single-Molecule Protein Identification Using Specific Amino Acid Signatures.

Subsection 1.1. Biochemistry Methods for High-Efficiency Labelling of Specific Amino Acids with DNA Barcodes, in Intact Proteins.

In this subsection, described herein is the employment of mature, highly-specific biochemistry reactions (e.g. NHS ester for lysine, maleimide for cysteine, EDC for acidic amino acids) for amino acid specific labelling of DNA barcodes [FIG. 5a, 5c]. Specifically, described herein is the development of a click chemistry-mediated two-step labelling method [FIG. 5b] to optimize for high-efficiency (>90%) labelling (i) with large pendant group (DNA strands, e.g., 10-20 nt in length), and (ii) in intact protein with dense labelling. The two-step method decouples labile crosslinker or intermediate from the slow reaction kinetics of the large DNA group. Strong denaturants (e.g. urea, guanidine, SDS) can be used to disrupt protein tertiary structure, and high salt to balance the charge density on DNA strands. Commercially available, sequence-defined model proteins (e.g. cytochrome C, RNase A) can be used for developing this method, and gel electrophoresis can be used for accurate readout of DNA-protein labelling efficiency. The sequence-dependent labelling efficiency can be further analyzed by digestion-based peptide mapping with a non-overlapping enzyme (e.g. GluC for lysine labels, trypsin for cysteine). These results can be used for both (i) providing feedback for optimization of the labelling methods, and (ii) establishing a sequence-based error model in a Bayesian based computational analysis framework (Subsection 1.4).

For extracting multiple amino acid signatures, described herein is the development of a high-efficiency and high-specificity labelling method for multiple labelling, using one of two alternative strategies: (i) serial labelling followed by purification after each step, (ii) parallel labelling with orthogonal click chemistry handles. Two fast, orthogonal click chemistry pairs have been reported (Saito, Noda, and Bode 2015). To further extend the range of accessible amino acid signatures, test methods can also be tested based on a few recent reports for specific labelling of additional amino acids (e.g. methionine, tyrosine and tryptophan (Lin et al. 2017, Ban et al. 2010, Antos et al. 2009)) [FIG. 5c]. Cross-linker synthesis can either be performed in-house following published protocols or outsourced. Their labelling efficiency and specificity can similarly be tested using model proteins, and use gel electrophoresis and peptide mapping to assay global labelling efficiency and local sequence dependence, respectively.

These data show that a two-step labelling scheme with NHS-DBCO crosslinker allows for high-efficiency (>95%) lysine labelling with DNA strands, on five model proteins [FIG. 6]. This result establishes that such high-efficiency and high-crowdedness DNA labelling on protein surfaces can be achieved under the right conditions.

Compatibility and potential crosstalk between different amino acid crosslinkers: To minimize labelling crosstalk, described herein is the design of the multi-labelling workflow (a) in the order of decreasing nucleophile reactivity, (b) as guided by their respective sequence-specific labelling efficiency and off-target reactivity, as obtained from peptide mapping. In the case that high-density DNA strands interfere with amino acid accessibility for succeeding labelling rounds, different denaturing buffer and high salt conditions can be tested, and parallel labelling workflow can be developed using orthogonal click chemistry handles.

Recently reported amino acid labelling chemistries may not be as specific as the traditional ones. Above a panel of three promising candidates has been identified and their individual performance can be tested. At least one candidate can be identified that is of high quality and compatible with the others, after optimization of conditions.

Subsection 1.2 A Single-Molecule Microscopy Method for High-Accuracy Amino Acid Counting and Protein Identification.

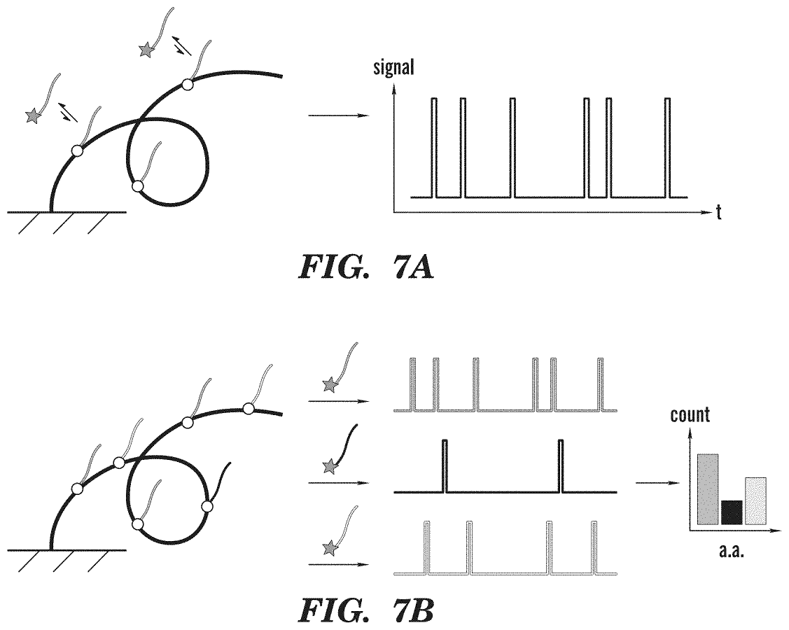

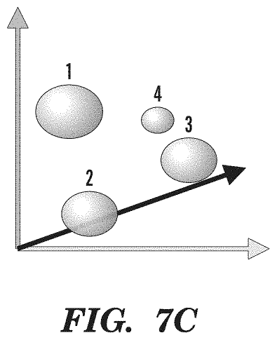

In this section, described herein is the development of a method for high-accuracy amino acid counting based on high-accuracy quantitative DNA-PAINT (qPAINT) imaging method (Jungmann et al. 2016) [FIG. 7a]. Specifically, DNA-barcoded protein samples prepared from Subsection 1.1 is first immobilized on a glass surface, using N- or C-terminus specific labelling chemistry (e.g. 2PCA label for N-terminus (MacDonald et al. 2015), or oxazolone for C-terminus (Yamaguchi et al. 2006)) and biotin-streptavidin linkage. Proteins with blocked N-terminus can be pre-treated chemically or enzymatically (Hirano and Kamp 2003) to expose the free amino group. To avoid protein aggregation and non-specific surface adsorption, the glass surface can be passivated, e.g., with protein blocking (e.g. BSA, casein) or PEG coating (Roy et al. 2008), and dilute the samples to appropriate concentration in the presence of surfactants. Then, single-molecule DNA-PAINT imaging can be performed under total internal reflection (TIR) illumination, for high-throughput, high-accuracy (<5% error) amino acid counting on single molecules. Different amino acids labelled with orthogonal DNA barcodes can be imaged either sequentially, using buffer exchange (Exchange-PAINT), or simultaneously with spectrally separable fluorophores (Jungmann et al. 2014) [FIG. 7b]. Assuming an average 100 nm separation between surface anchored protein molecules, 50×50 um field-of-view allows >2×10⁵ molecules to be imaged at the same time.

First model proteins from Subsection 1.1 can be used to develop this method and assay the single-molecule counting accuracy, precision, for each of the amino acid labels. The effect of different DNA sequences and imaging conditions (e.g. salt, surfactants) can be tested to optimize the counting performance. The linearity between the results and the expected amino acid abundance can be assayed, in different model proteins, and correct for any systematic effects. Any potential sequence-based bias in terminal labelling and surface anchoring efficiency can also be characterized. Then, the coverage of different types of proteins (e.g. intrinsically disordered proteins, membrane proteins, large proteins) can be extended from either commercial sources, or in-house protein expression, and the uniformity of in surface deposition and counting accuracy can be assayed, and any systematic effects can be characterized. Finally, the method can be tested with complex protein samples from either expressed protein library (e.g. kinome library (Gujral et al. 2014)), or organelle lysate samples (e.g. mitochondria (Rhee et al. 2013)). High-throughput (>10⁵) amino acid counting can be performed on single molecules, followed by protein identification by matching multiple amino acid abundances [FIG. 7c]. The identification accuracy and dynamic range of the method can be validated by comparing against previous organelle proteome maps (Thul et al. 2017, Itzhak et al. 2016).

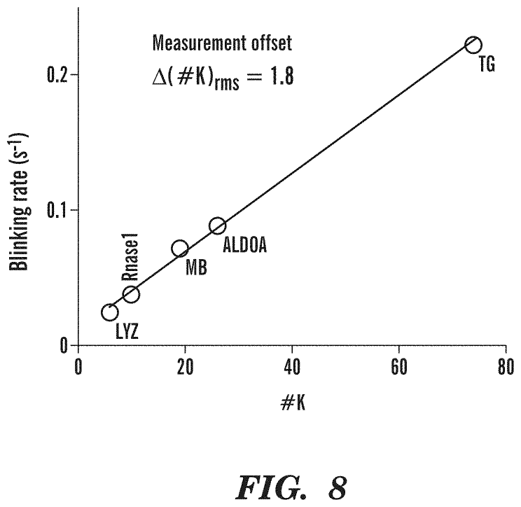

The data using 2PCA-based N-terminal labelling has shown specific labelling on model proteins [FIG. 8]. After DNA barcode labelling and surface deposition, DNA-PAINT imaging allows high-accuracy kinetic analysis on single molecules, correctly reporting the lysine content on a panel of five model proteins. Although further optimization can be beneficial, these results establish the foundation (for both Subsections 1.2 and 1.3) of single-molecule DNA-PAINT imaging on surface-anchored proteins, and the feasibility of accurate DNA-PAINT imaging.

The dense DNA labels and hydrophobic protein core, exposed after denaturation (esp. in membrane proteins), may present a different microenvironment that interferes with uniform accessibility of imager strands and prevents accurate counting. Non-specific adsorption of DNA barcodes and protein backbone to glass surface could further contribute to the problem. To address these problems, a few potential solutions can be tested: (a) use a hydrophilic linker (e.g. PEG) for DNA labelling, (b) develop new conditions for DNA-PAINT imaging (e.g. with denaturants), (c) use DNA-analogues (e.g. PNA) that have different charge and kinetic parameters, and combine them if necessary. In addition, different surface treatment (e.g. covalent attachment, or one-step silane modification) can be employed to change the surface charge profile and reduce undesired DNA-surface interaction, as well as allow more flexible imaging conditions.

The high-density DNA labels could also bias DNA-PAINT kinetics and interfere with accurate counting (e.g. sub-linear behavior), due to “hopping” between nearby DNA barcodes. This problem can be addressed either by modelling any systematic effects and correct for it during analysis, or developing an exchange-based sub-division strategy (i.e. use multiple DNA barcodes per each amino acid) to reduce the effective DNA strand density and restore accurate counting.

Subsection 1.3 A Biophysical Method for Protein Backbone Extension, and a Single-Molecule Microscopy Method for Amino Acid Linear Barcoding and Protein Identification.

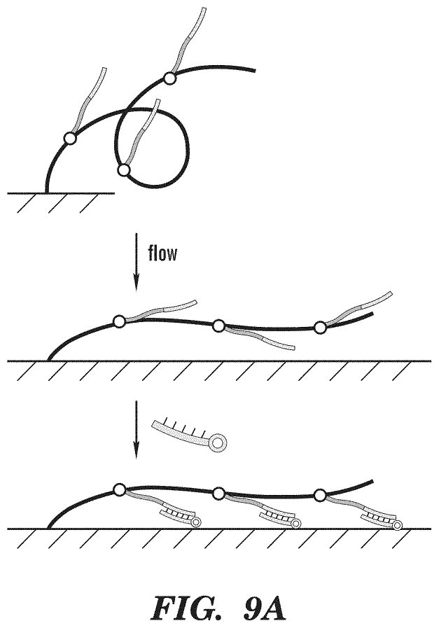

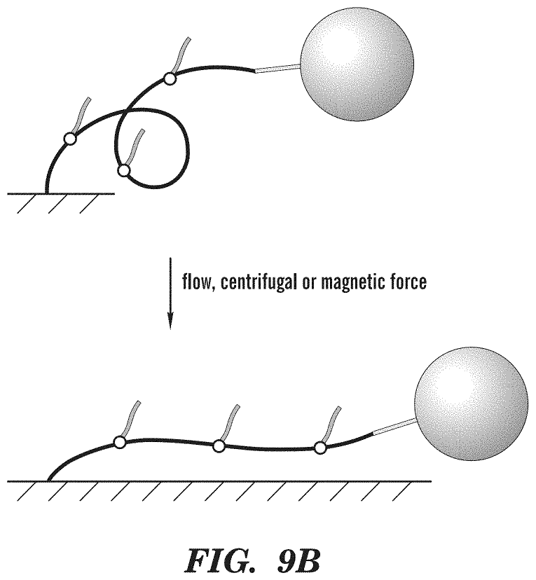

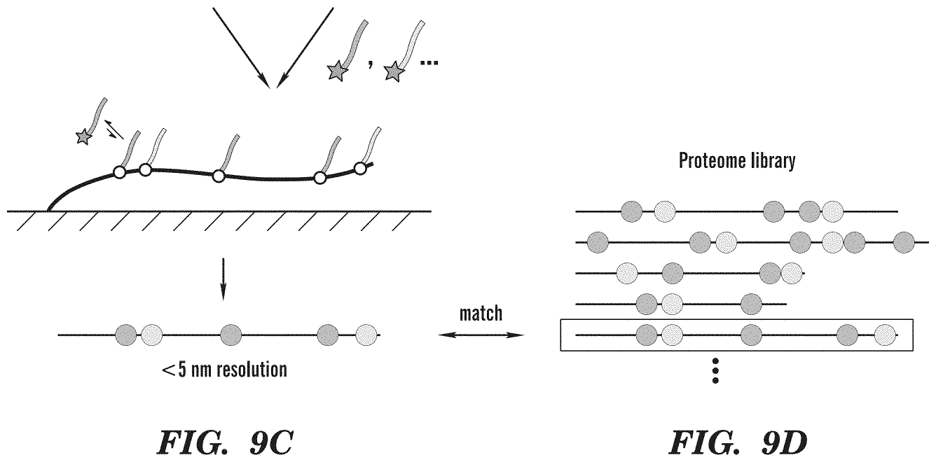

In this section, described herein is the development of a biophysical method for protein backbone extension, that allows robust protein identification with amino acid linear barcoding. Specifically, two alternative approaches are developed. (i) Stretching by hydrodynamic flow [FIG. 9a]. The theoretical estimate discussed above shows that, with dense DNA labelling, hydrodynamic drag force generated with high viscosity buffer is enough to extend protein backbone, e.g., to >80% contour length. Specifically, first DNA labelling and surface anchoring of protein samples can be performed as developed in Subsection 1.2, in micrometer-sized channels, and then high-speed flow controlled by a syringe pump is introduced, with a high-viscosity buffer (e.g. with glycerol). The extended protein backbone can be anchored on surface by introducing an anchoring strand (Geiss et al. 2008), that comprises a sequence complementary to the DNA barcodes, and a surface-binding group. (ii) Stretching by micro-bead [FIG. 9b]. In this approach, specific labelling can be performed on the protein's C-terminus and attach it to a micro-bead, optionally through a long linker to accommodate the bead's size. The micro-bead can then be stretched via flow, centrifugal (Halvorsen et al. 2010), or electromagnetic force (Strick et al. 1996). As compared to approach (i), approach (ii) allows the application of controllable, and higher pulling force on the protein. After backbone extension, high-sensitivity and high-resolution DMI imaging (Dai et al.2016) can be applied to read out amino acid linear signatures [FIG. 9c, 9d].

The method can first be developed with a moderate-sized (10-20) library of large model proteins (100+kDa) to facilitate observation and characterization of protein extension. For approach (i), organic dyes can be used to label the protein samples along with the DNA barcodes, to facilitate real-time visualization of protein backbone during flow stretching. For approach (ii), tracking the bead position allows real-time and high-precision measurement of protein extension. After protein backbone extension, the global stretching efficiency (as a fraction of contour length) can first be assayed by DNA-PAINT super-resolution imaging, and optimize the above conditions as necessary. The super-resolution images can then be compared against expected amino acid linear signatures, assay stretching efficiencies across different parts of the backbone, and test for any local, sequence- or DNA density-dependent effects. Next, this method can be developed for complex protein samples (as in Subsection 1.2). For this subsection, protein identification can be performed with a simplified algorithm (more sophisticated algorithm to be developed in Subsection 1.4). Identification accuracy and dynamic range can be similarly assayed as in Subsection 1.2.

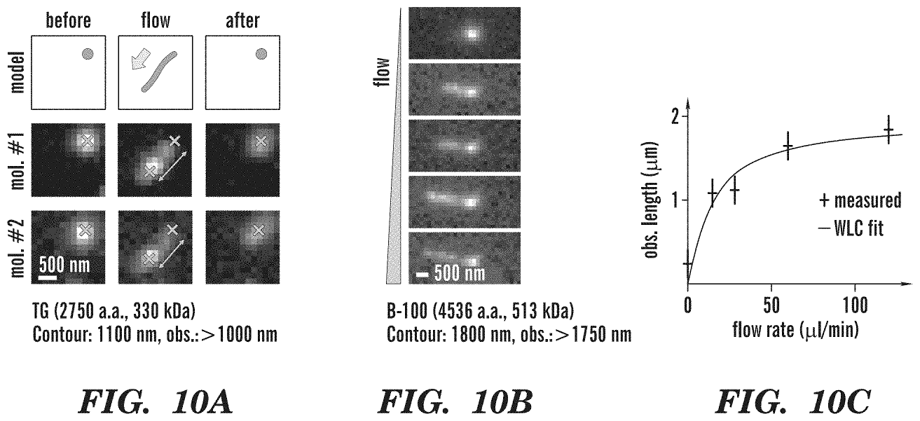

These data show that hydrodynamic drag allows backbone extension of DNA-labelled model proteins thyroglobulin (330 kDa) and apolipoprotein B (513 kDa) to >90% of their contour length [FIG. 10], under high-viscosity buffer and high flow, with a force-extension behavior consistent with worm-like chain (WLC) model. These results establish that dense DNA labelling can indeed increase the hydrodynamic drag exerted on protein backbone, allowing for faithful amino acid linear barcoding and accurate protein identification in complex mixtures.

For potential problems introduced by non-specific protein- or DNA-surface interaction, the same strategies above (Subsection 1.2) can be applied.

Potential incomplete and non-uniform protein backbone extension could result from a few likely causes: (a) Due to its cumulative nature, the stretching force will be reduced close to the tail of the protein backbone. Conditions can be optimized to minimize this effect, and a correction algorithm can be developed for this effect in the analysis workflow. (b) Certain protein secondary or tertiary structures may be harder to extend fully (e.g. certain beta barrel folds require higher unfolding force (Dietz and Rief 2004)). The sequence- or fold-dependence in stretching defects can first be analyzed from the model protein library, and optimize conditions accordingly (e.g. with different chemical denaturants (Parui and Jana 2019)). A machine learning based analysis algorithm can then be developed to predict and correct for sequence-dependent extension defects. (c) The median length of a human proteins is ˜400 a.a. (i.e. ˜150 nm when fully stretched). To avoid overlap and allow faithful readout of the extended linear signatures, a larger per-molecule footprint is necessary during uncontrolled deposition, which translates to lower surface density (as compared to Subsection 1.2) and profiling throughput. Microbead-based extension approach requires even larger foot-print due to the size of the bead. To address this problem, lithography-based surface patterning (Deufel et al. 2007) can be employed for regular, higher-density protein anchoring. In addition, DMI based imaging methods can be optimised and shortened as compared to that required for high accuracy counting, improving the overall throughput.

Subsection 1.4 A Computational Analysis Platform for Robust Single-Molecule Protein Assignment and Single-Cell Proteomic Analysis.

In this section, described herein is the development of an algorithm for robust single-molecule protein identification, from amino acid signatures measurements obtained in Subsections 1.2 and 1.3. The identification algorithm can proceed in three steps: (i) super-resolution analysis of microscopy images, (ii) identification and isolation of single molecules, and (iii) amino acid signature extraction and library matching. The analysis Table 1 can be built on, and adapted from established strategies in sequence alignment and mass spectrometry proteomic analysis. In details, the algorithm can first construct an error model, incorporating any systematic (e.g. sequence-dependent bias in labelling efficiency or backbone extension) and stochastic effects (e.g. missing labels, off-target labels) determined from Subsections 1.1 and 1.3. During library search, the observed signature can be compared with all possible readouts from all library proteins, generated using the above error model. The final identification can be performed with a Bayesian framework, further incorporating any prior bias in surface anchoring efficiency and proteome abundance, and gated by false discovery rate (FDR), to allow for robust identification in complex mixtures.

Next, algorithms can be developed for single-cell proteomic analysis and cell state classification. The algorithm can be adapted from single-cell transcriptomic analysis (Klein et al. 2015, Weinreb et al. 2017), and can operate in four steps: data scaling and z-score normalization, dimensionality reduction (using tSNE or UMAP), distance-based clustering, and expression analysis within and across clusters. Two adaptations can be made for single-molecule proteomics analysis: (i) Since the method observes intact proteins rather than peptide fragments, different protein isoforms will be separately identified, but grouped together for clustering analysis. (ii) protein-specific expression noise models (Bar-Even et al. 2006, Pedraza and Paulsson 2008) can be incorporated, especially for low-copy proteins. These algorithms can be developed and validated using experimental data obtained from Subsections 1.2 and 1.3.

Complex proteome samples, especially non-standard protein isoforms and truncations, may interfere with accurate identification. References can first be incorporated from different sources and methods (e.g. SwissProt, TrEMBL, and potentially transcriptomic-derived references (Wühr et al. 2014)), and false discovery rate (FDR) controlled search strategies can also be incorporated (e.g. target-decoy analysis), to ensure accurate and robust identification.

High-density DNA labels may interfere with discrete molecular fitting (after super-resolution image reconstruction). To address this problem, the following can be done: (a) apply multi-emitter fitting algorithms (Zhu et al. 2012), and/or (b) combine blinking kinetics data with spatial localization to help determine the multiplicity of overlapping emitters and improve identification confidence.

Outcome for Section 1.

With successful completion of Section 1, a workflow is established for high-throughput, single-molecule protein identification, by accurate optical readout of specific amino acid signatures. Subsection 1.1 provides biochemistry methods for converting specific amino acids to DNA barcodes in intact proteins; Subsections 1.2 and 1.3 provide two complementary methods for accurate, single-molecule readout of these DNA barcodes, by measuring their abundance and linear distribution along the protein backbone, respectively; subsection 1.4 further provides a computational method for robust protein identification in complex mixtures.

Section 2. A Microfluidic Workflow for Single-Molecule Protein Identification and Single-Cell Proteomics

In this section, described herein is the development of a microfluidic workflow for single-cell proteomics, comprising four steps: (i) on-chip single-cell lysis and protein capture, (ii) surface-based specific amino acid labelling with DNA barcodes, (iii) accurate single-molecule DNA-PAINT imaging for amino acid signature readout, and (iv) data analysis for protein identification and single-cell proteomics. Previous microfluidic platforms for single-cell time-lapse imaging, optical isolation, and on-chip lysis can be built upon (Potvin-Trottier et al. 2018, Wang et al. 2018). In particular, a method for non-targeted surface protein capture and specific amino acid labelling (Subsection 2.1) can first be developed and then combined with previous methods into an integrated experimental workflow for on-chip single-cell lysis, protein labelling and single-molecule imaging (Subsection 2.2).

Subsection 2.1 A Method for Non-Targeted Surface Protein Capture and Specific Amino Acid Labelling Compatible with Microfluidic Device.

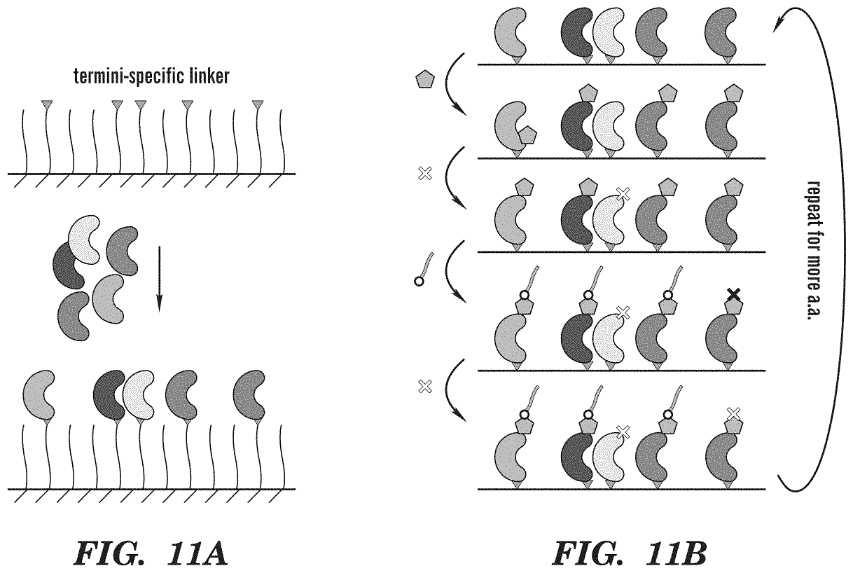

In this section, protein samples are first anchored onto glass surface that has been pre-treated with N- or C-terminus-specific labelling moiety (e.g. 2PCA (MacDonald et al. 2015), or oxazolone label (Yamaguchi et al. 2006)) [FIG. 11a]. For N-terminus anchoring, the proteins can be incubated with N-deblocking aminopeptidase (e.g. Pfu) beforehand, to free up the N-terminus. This can allow covalent protein-surface binding that is resistant to strong denaturing and organic conditions necessary in down-stream steps. Next, amino acid specific labelling (e.g. on lysine, cysteine, and the acidic amino acids) can be performed with click chemistry mediated two-step labelling method, as introduced in Subsection 1.1, to ensure high-efficiency labelling. To minimize off-target labelling, multiple amino acid labelling can be performed in order of decreasing reactivity, and unreacted amino acids or crosslinker can be capped by non-reactive, small molecule quenchers after each step [FIG. 11b].

This protocol can be first developed with a moderate-sized (10-20) library. Labelling efficiency and specificity for each amino acid will be assayed by qPAINT imaging. Then surface capture and labelling conditions can be optimized (e.g. surface linker length, denaturing buffer, pH) to improve solubility and labelling efficiency at difficult sequence regions. Finally, the method can be applied to complex protein samples and assay identification accuracy and dynamic range (as in Subsection 1.2).

A potential problem is incompatibility between reaction conditions that allow high-efficiency amino acid labelling and those that minimize non-specific protein-surface interaction. A few potential solutions can be tested, including (a) varying the length, charge and hydrophobicity of surface linker (e.g. PEG, DNA and DNA analogues, polysaccharide linkers), (b) using different surface passivation methods (e.g. protein blocking, PEG, surfactants) and tuning surface charge layer, (c) performing labelling reactions in organic phase instead, that could allow high amino acid accessibility and disrupt non-specific surface adsorption.

Unspecific background labelling on glass and PDMS surface could interfere with accurate single-molecule imaging. To minimize such background, one-step silane chemistry can be used for glass surface modification (Gidi et al. 2018) to avoid introducing reactive amino groups. PDMS surface can also be passivated with previously reported methods (Huang et al. 2007, Huang et al. 2005) to reduce undesired surface labelling.

Subsection 2.2 An Integrated Microfluidic Workflow for Single-Cell Lysis, Protein Capture, and Single-Molecule Proteomic Profiling.

For this section, the surface protein capture and labelling method developed in Subsection 2.1 can be integrated with previously reported microfluidic single-cell handling methods, to develop an integrated microfluidic workflow for single-cell lysis, protein capture and proteomic profiling. The workflow can operate in four steps: (i) single-cell on-chip lysis and protein capture, (ii) surface-based DNA labelling on specific amino acids, (iii) single-molecule DNA-PAINT imaging, and (iv) protein identification and single-cell analysis. Specifically, single cells can be first diluted to a low surface density, or isolated into individual micro-wells equipped with pressure-controlled valves, and then lysed on-chip with one of four methods: mechanical rupturing, electroporation, chemical or enzymatic treatment (Nan et al. 2013). Proteins released from individual cells can be captured on pre-treated glass surface (Subsection 2.1), and separated either by diffusion-limited deposition (Wang et al. 2018) or inside closed microwells (Shi et al. 2012), ready for subsequent labelling and single-molecule imaging.

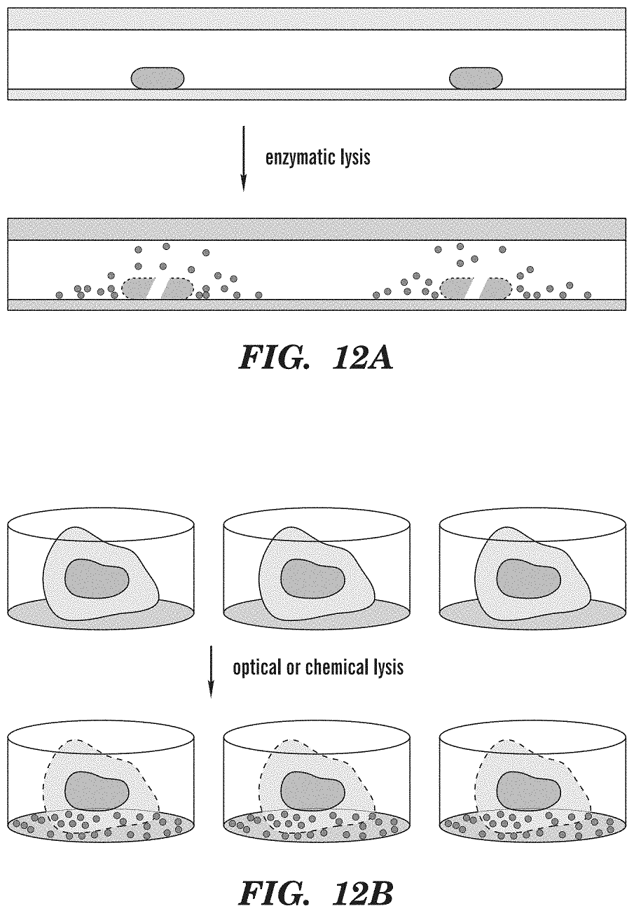

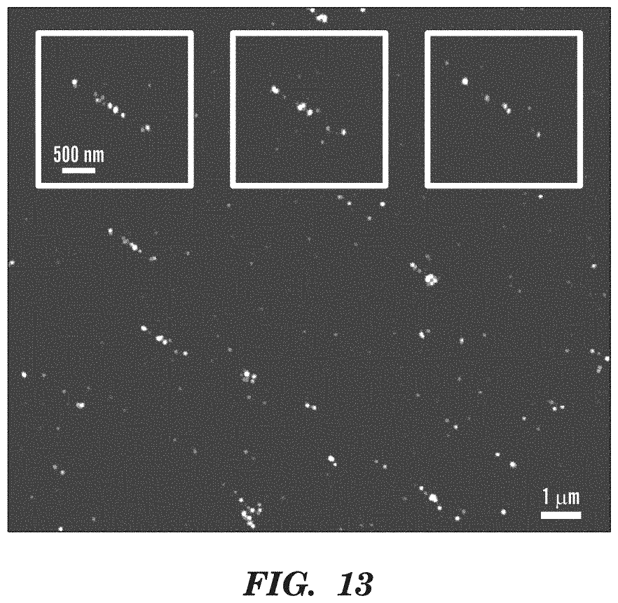

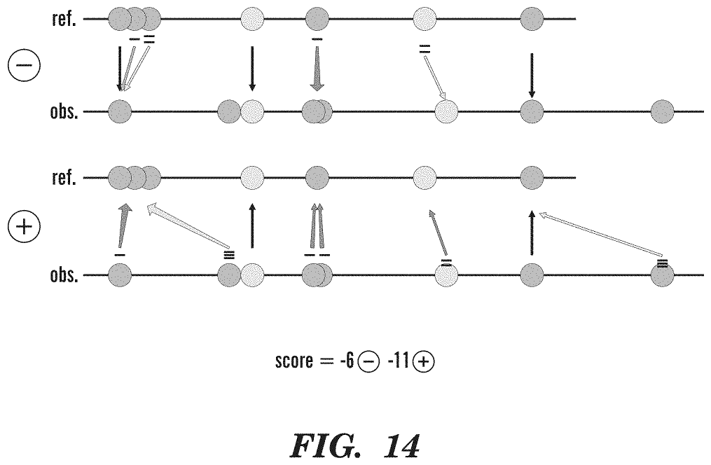

Two variations of these methods can be developed, for bacterial and mammalian cells, respectively. For bacterial cells, enzymatic or chemical cell lysis can be used, combined with diffusion-limited protein capture in open channels [FIG. 12a]; for mammalian cells, a micro-well based cell lysis method can be adapted using chemical, mechanical or optical treatment [FIG. 12b]. These methods can be developed using model cell lines with well-characterized reference proteome and deep proteomic data (e.g. E. coli K-12 MG1655 strain (Schmidt et al. 2015), HeLa CCL-2 (Nagaraj et al. 2011) and Jurkat cell lines (Geiger et al. 2012)). The protein capture and identification efficiency can be assayed by performing correlated live-cell imaging (Luro et al. 2020) and single-cell proteomics with fluorophore-tagged test proteins (Lepore et al. 2019, Okumus et al. 2016), as well as compare the results against previous deep proteomic studies. The method can be further validated by comparing single-cell proteomic profiles under normal condition and metabolic stress. Finally, an intermediate-scale single-cell proteomics study can be performed on 100 isolated bacterial cells, and proteomics-based cell state analysis can be performed using algorithms developed in Subsection 1.4.

A high concentration of lipids, sugar and nucleic acids released from the cell and salt from chemical lysis buffer may interfere with efficient and specific protein capture on surface. For this reason, to the following are preferred methods: non-chemical lysis methods (e.g. enzymatic for bacteria (Wang et al. 2018), mechanical or optical for mammalian cells (Nan et al. 2013)). In addition, the cellular content can be diluted by a large factor (>100×) in volume after lysis.

Single-cell protein capture throughput is limited by the density of protein-capturing groups, and may not provide enough profiling depth. To address this concern, the surface capturing density can be optimized and the available surface area adapted for each cell by controlling their spatial separation (for bacteria), or designing micro-wells (for mammalian cells). With appropriate spacing (˜100 nm, see Subsection 1.2) between protein anchors, a detection throughput of 2×10⁵ per imaging session is expected, which is enough to represent 10% of all proteins in an E. coli cell (bionumbers.org), allowing for deep proteomic profiling in single cells (detection limit 10 copies). For human cells, although 2×10⁵ proteins is a much lower fraction (0.1%) of the cellular protein content (Wiśniewski et al. 2014), it still allows very sensitive detection down to 1,000 copies per cell, offering an effective in-depth proteomic analysis (Zubarev 2013) in single human cells.

Outcome for Section 2.

In Section 2, described herein is the development of a microfluidic workflow for single-molecule based single-cell proteomics, for bacterial and mammalian cells. Subsection 2.1 can establish a surface-based protein capture and amino acid labelling method; Subsection 2.2 can develop it into an integrated single-cell proteomics workflow. This workflow can be further combined with existing techniques into an integrated platform for single-cell lineage tracking, time-lapse imaging and targeted proteomic profiling.

TABLE 1
Human proteome coverage. Amino acid signature allows accurate
identification of human proteome. Source: UniProt
UP000005640_9606, longest proteins. Top hit: protein is correctly
identified by highest-scoring candidate; FDR 2%: score-gated
proteomecoverage set at 98% accuracy.

| | |
|---|---|
| K only | K + C |

| Conditions | Top hit | FDR 2% | Top hit | FDR 2% |
|---|---|---|---|---|
| error-free/5 nm | 95.8% | 96.5% | 98.0% | 100% |
| 20% err./5 nm | 85.3% | 78.6% | 92.9% | 92.3% |
| 20% err./10 nm | 68.1% | 51.8% | 82.8% | 75.8% |

1. A method for obtaining partial sequence information from a target protein, comprising
a) denaturing a protein;
b) labeling occurrences of one or more particular amino acids in the protein;
c) capturing the protein on a substrate via its N-terminus or C-terminus;
d) elongating the protein; and
e) imaging the substrate to detect labeled amino acids, thereby locating the particular amino acids in the protein, whereby partial sequence information is obtained for the target protein.

2. The method of claim 1, wherein labeling occurrences of one or more particular amino acids comprised fluorescent labeling.

3. A method for obtaining partial sequence information from a target protein, comprising
a) denaturing a protein;
b) attaching docking strands to particular amino acids in the protein;
c) capturing the protein on a substrate via its N-terminus or C-terminus;
d) elongating the protein;
e) repeatedly contacting the captured protein with fluorescently-labeled imager strands that transiently bind to respective docking strands attached to particular amino acids in the protein; and
f) imaging the substrate, thereby locating the particular amino acids in the protein, whereby partial sequence information is obtained for the target protein.

4. The method of claim 3, wherein the docking strands and imager strands comprise nucleic acid strands.

5. The method of claim 1, wherein the step of capturing the N-terminus of the protein of the substrate comprises contacting the N-terminus of the protein with a cross-linking agent comprising 2-Pyridinecarboxaldehyde (2PCA).

6. The method of claim 5, wherein a cross-linking agent is Tetrazine-2-Pyridinecarboxaldehyde (TZ-2PCA).

7. The method of claim 5, wherein the cross-linking agent specifically reacts with a moiety on the substrate.

8. The method of claim 7, wherein the moiety on the substrate comprises trans-cyclooctene (TCO).

9. The method of claim 1, wherein the step of capturing the C-terminus of the protein of the substrate comprises contacting the C-terminus of the protein with a cross-linking agent comprising oxazolone.

10. The method of claim 1, wherein the step of elongating the protein comprises microfluidic elongation in a microfluidic device.

11. The method of claim 10, wherein a microfluidic channel of the microfluidic device is at least 10 μm in width.

12. The method of claim 10, wherein the microfluidic elongation comprises flowing fluid past the protein at a flow rate of at least 20 uL/min.

13. The method of claim 10, wherein the fluid has a viscosity of at least 1.4 Pa·s.

14. The method of claim 10, wherein the fluid comprises glycerol.

15. The method of claim 10, wherein the fluid comprises a denaturant.

16. The method of claim 15, wherein the denaturant is selected from the group consisting of urea, guanidine, and sodium dodecyl sulfate (SDS).

17. The method of claim 1, wherein the step of elongating the protein comprises:
a) linking the N-terminus of the protein to a first substrate, and linking the C-terminus of the protein to a second substrate; or
b) linking the C-terminus of the protein to a first substrate, and linking the N-terminus of the protein to a second substrate.

18. The method of claim 17, wherein the first substrate comprises a surface in a microfluidic device.

19. The method of claim 17, wherein the second substrate is a microbead.

20. The method of claim 17, further comprising applying a fluid flow force, centrifugal force, or magnetic force to the second substrate.

21. The method of claim 1, wherein the protein is elongated to at least 80% of its expected contour length.

22. The method of claim 1, further comprising the step of: determining a score for an observed pattern of amino acid labeling compared to an expected pattern of amino acid labeling.

23. The method of claim 22, wherein partial sequence of the protein is determined if the score is above a pre-determined threshold.

24. A system comprising:
a) a substrate;
b) a protein cross-linked to the substrate via its N-terminus or C-terminus;
c) docking strands attached to particular amino acids in the protein; and
d) fluorescently-labeled imager strands that transiently bind to docking strands attached to particular amino acids in the protein.

25. A microfluidic device comprising:
a) a cross-linking reagent;
b) docking strands attached to particular amino acids in a protein;
c) fluorescently-labeled imager strands that transiently bind to docking strands attached to particular amino acids in a protein; and
d) a high-viscosity and/or denaturing buffer.

26. A kit comprising:
a) a substrate;
b) a cross-linking reagent that permits attachment of a protein to the substrate;
c) docking strands comprising a functional group permitting attachment to particular amino acids in a protein;
d) fluorescently-labeled imager strands that transiently bind to respective docking strands; and
e) a high-viscosity and/or denaturing buffer.

27. The system of claim 24, wherein the docking strands and imaging strands comprise nucleic acid strands.
